(19) United States
(12) Patent Application Publication
(10) Pub. No.: US 2024/0261554 A1
(43) Pub. Date: Aug. 8, 2024

(54) Intraosseous Hemodialysis Port

(71) Applicant: Bard Access Systems, Inc., Salt Lake City, UT (US)

(72) Inventors: Oladipo P. Akerele-Ale, Phoenix, AZ (US); Ericka J. Prechtel, Salt Lake City, UT (US)

(21) Appl. No.: 18/105,533

(22) Filed: Feb. 3, 2023

(51) Int. Cl. A61M 39/02; A61M 1/14

(52) U.S. Cl. CPC A61M 39/0208; A61M 1/14

(57) ABSTRACT

Various embodiments of an intraosseous port configured for subcutaneous placement within a patient. Some embodiments include a pair of cavities, each cavity including a septum for receiving a needle. Other embodiments include a graft extending between the cavities. Each cavity is coupled with a lumen extending into a cavity of a bone during use, e.g., a medullary cavity. A method includes extracting a bodily liquid from the bone cavity and returning the bodily liquid back to the bone cavity. Some methods include performing hemodialysis via the intraosseous port.

### BACKGROUND

Vascular access devices are widely used to deliver a wide variety of substances, including fluids, medications including chemotherapy, blood products, and total parental nutrition. Transcutaneous vascular access devices include standard central venous catheters (CVC), tunneled central venous catheters and peripherally inserted central venous catheters (PICC). These catheters pass through the skin, enter a vein and terminate in a central venous location. Transcutaneous VADs can be left at the insertion site for weeks or more, and as such require regular flushes with saline and/or an anti-coagulant solution to protect against thrombus formation and occlusion. implantable VADs are used in patients who require access for weeks to months. The implantable VADs typically have a metal or plastic port that is implanted into the subcutaneous tissue and anchored to the fascia tissue along with a catheter portion that enters a vein. The implantable VADs also require regular flushes with anti-coagulant solution, typically concentrated heparin to prevent thrombus formation and occlusion.

Currently available VADs bear significant risk (about 10%) of introducing infection to a blood stream, which can lead to serious costly complications such as bacteremia, 45 sepsis or even death. Furthermore, because they are in constant contact with the blood stream, the VADs require regular flushes to clear stagnant blood and prevent thrombus formation and occlusion. Even with regular flushes occlusions occur in approximately 30% of patients, requiring treatment with thrombolytic agents or device removal and reinsertion of a new device, which are costly, can interfere with patient care and result in complications.

Accordingly, there is a need for vascular access devices that enable exchanging fluids with patients that are easy to implant and access, and which are less prone to occlusion and the various limitations outlined above.

### SUMMARY

Disclosed herein is an intraosseous port that, according to some embodiments, includes a frame configured for subcutaneous placement within a patient. The frame defines a proximal portion having first cavity and a second cavity, where the first and second cavities are configured for fluidly coupling with a medical system. The frame also defines a distal portion configured for insertion through a bone wall, where the distal portion includes (i) a first lumen extending along the distal portion between the first cavity and open distal end of the first lumen and (ii) a second lumen extending along the distal portion between the second cavity and an open distal end of the second lumen, where the first and second open distal ends are configured for placement within a cavity of the bone. The intraosseous port further includes an anchoring mechanism configured for securing the frame to the bone.

In some embodiments, the open distal end of the second lumen is spaced longitudinally away from the open distal end of the first lumen. In some embodiments, the second cavity is disposed laterally with respect to the first cavity, and in some embodiments, the second lumen is disposed laterally with respect to the first lumen.

In some embodiments, the anchoring mechanism includes external threads, and in some embodiments, the anchoring mechanism includes a first threaded portion surrounding the first lumen and a second threaded portion surrounding the second lumen.

In some embodiments, the intraosseous port further includes a first septum extending across a proximal opening of the first cavity and a second septum extending across a proximal opening of the second cavity, where the first and second septa are configured for passage of a needle therethrough.

In some embodiments, the intraosseous port further includes a graft extending between the first and second cavities. In some embodiments, the graft is configured to extend between the bone and an under surface of a skin of the patient. In some embodiments, the bone wall includes a greater tubercle of a humerus.

Also disclosed herein is method of treating a patient that, according to some embodiments, includes (i) coupling an intraosseous port to a bone of the patient, (ii) establishing a fluid connection between a medical system and the intraosseous port, (iii) extracting a first bodily liquid from the patient via the intraosseous port, and (iv) delivering a second bodily liquid to the patient via the intraosseous port.

In some embodiments of the method, coupling an intraosseous port to a bone of the patient includes implanting the intraosseous port subcutaneously within the patient.

In some embodiments of the method, extracting a first bodily liquid includes drawing the first bodily liquid from a cavity of the bone via a first lumen of the intraosseous port, and delivering a second bodily liquid includes delivering the second bodily liquid to the cavity of the bone via a second lumen of the intraosseous port.

In some embodiments of the method, extracting the first bodily liquid and delivering the second bodily liquid occur simultaneously.

In some embodiments of the method, the second bodily liquid includes the first bodily liquid in a processed state in accordance with an operation of the medical system.

In some embodiments of the method, coupling an intraosseous port to a bone of the patient includes threading the intraosseous port into the bone.

In some embodiments of the method, establishing a fluid connection between a medical system and the intraosseous port includes (i) piercing a first septum of the intraosseous port with a first needle and (ii) piercing a second septum of the intraosseous port with a second needle.

In some embodiments of the method, the intraosseous port includes a graft extending between the first lumen and the second lumen, and establishing a fluid connection between a medical system and the intraosseous port includes piercing the graft with a first needle and piercing the graft with a second needle.

In some embodiments of the method, the medical system includes hemodialysis system. In some embodiments, extracting the first bodily liquid includes defining a flow rate of the first bodily liquid that exceeds about 50 ml/min.

These and other features of the concepts provided herein will become more apparent to those of skill in the art in view of the accompanying drawings and following description, which describe particular embodiments of such concepts in greater detail.

### DRAWINGS

FIG. 1 illustrates a front elevation partial section view of a first embodiment of an intraosseous port in use with a patient, in accordance with some embodiments.

FIG. 2 illustrates a front elevation partial section view of a second embodiment of an intraosseous port in use with a patient, in accordance with some embodiments.

FIG. 3 illustrates a front elevation partial section view of a third embodiment of an intraosseous port in use with a patient, in accordance with some embodiments.

FIG. 4 illustrates a block diagram of a method for treating a patient via the intraosseous port, in accordance with some embodiments.

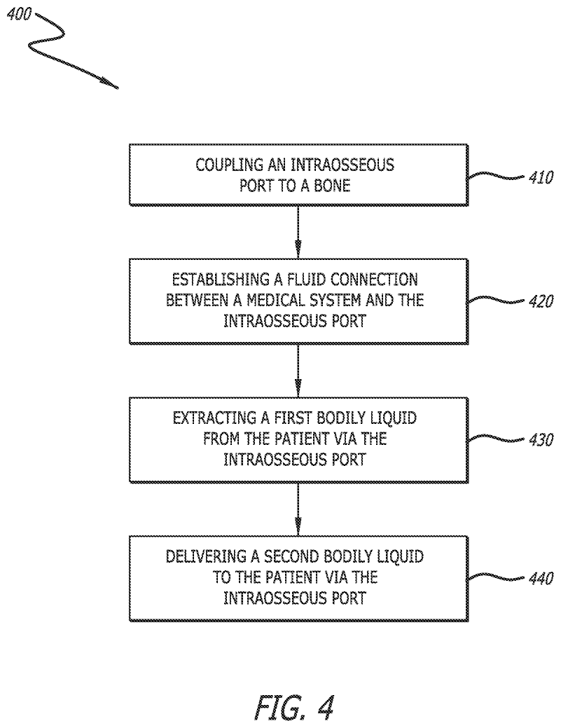

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein. Additionally, all embodiments disclosed herein are combinable and/or interchangeable unless stated otherwise or such combination or interchange would be contrary to the stated operability of either embodiment.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The phrases “connected to,” “coupled with,” and “in communication with” refer to any form of interaction between two or more entities, including but not limited to mechanical, electrical, magnetic, electromagnetic, fluid, and thermal interaction. Two components may be coupled with each other even though they are not in direct contact with each other. For example, two components may be coupled with each other through an intermediate component.

The terms “proximal” and “distal” refer to opposite ends of a medical device, including the devices disclosed herein. As used herein, the proximal portion of an implanted intraosseous port is the portion nearest the skin surface of the patient during use, while the distal portion is the portion furthest embedded within the patient body.

Unless defined otherwise, all technical and scientific terms used herein have the same meaning as commonly understood by those of ordinary skill in the art. References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

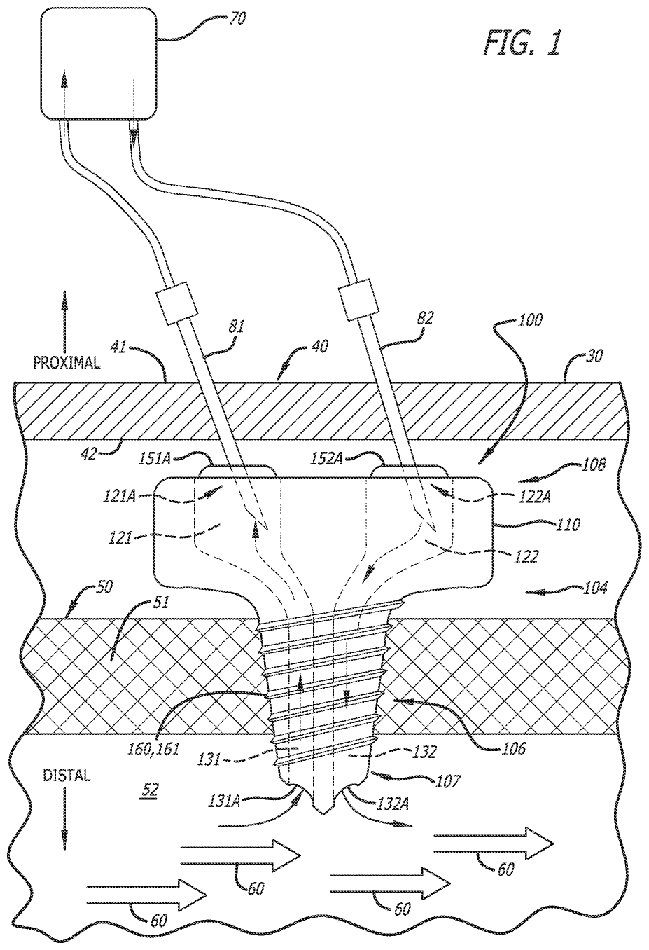

FIG. 1 illustrates a front elevation partial section view of a first embodiment of an intraosseous port (IOP) 100 in use with a patient during a treatment defined by a medical system (system) 70. The IOP 100 is generally configured to be implanted within the patient, such as entirely within patient, for example. The IOP 100 is further generally configured to enable access to a bodily liquid of the patient, such as blood, for example. In some embodiments, the system 70 may be configured to withdraw the bodily liquid (e.g., blood) from the patient and deliver the bodily liquid back to the patient in a processed state in accordance with operation of the system 70, such a hemodialysis system, for example. In other embodiments, the system 70 may be configured to withdraw the bodily liquid from the patient and deliver a different liquid to the patient. In the illustrated embodiment, the IOP 100 is configured to enable access to the bodily liquid within a bone, such as within a medullary cavity of the bone.

FIG. 1 shows the IOP 100 implanted within a patient 30 such that the IOP 100 is disposed entirely beneath the skin 40. The IOP 100 includes a proximal portion 104 disposed between an under surface 42 of the skin 40 and a bone 50. The IOP 100 is coupled with the bone 50 such that a distal portion 106 of the IOP 100 extends through a bone wall 51 of the bone 50. A distal end 107 of the IOP 100 is disposed within a cavity 52 of the bone 50, such as a medullary cavity, for example. In some embodiments, the IOP 100 may configured such that a proximal end 108 of the IOP 100 is disposed adjacent the under surface 42 of the skin 40.

The IOP 100 includes a frame 110 that generally defines the structure of the IOP 100. The frame 110 may be formed of any rigid material suitable for implantation within the patient 30, such as stainless steel or plastic, for example. The frame 110 defines a first cavity 121 fluidly coupled with a first lumen 131 to define a first flow path extending between a first cavity opening 121A at the proximal end 108 and a first lumen opening 131A at the distal end 107. Similarly, the frame 110 further defines a second cavity 122 fluidly coupled with a second lumen 132 to define a second flow path extending between a second cavity opening 122A the proximal end 108 and a second lumen opening 132A at the distal end 107. In the illustrated embodiment, the first cavity 121 and the first lumen 131 are positioned laterally with respect to the second cavity 122 and the second lumen 132 in side-by-side relationship. In other embodiments, the first cavity 121 including the first lumen 131 and second cavity 122 including the second lumen 132 may be positioned concentrically with respect to each other.

The IOP 100 includes a first septum 151A extending across the first cavity opening 121A and a second septum 152A extending across the second cavity opening 122A. The first and second septa 151A, 152A define a seal over the first and second cavity openings 121A, 122A, respectively. The first and second septa 151A, 152A are configured for piercing via a needles 81, 82 and resealing after the needles 81, 82 are removed. The first and second cavities 121, 122 are configured to receive tips of the needles 81, 82, respectively therein to provide access to the bodily liquid 60 therein. The first and second septa 151A, 152A may be formed of any suitable septum material configured for implantation within the body, such as EPDM, polytetrafluorethylene, Dacron, silicon, or polyurethane, for example. During use, the first and second septa 151A, 152A may be pierced several times (e.g., 5, 10, 20, 50, or more) over the life of the IOP 100.

The first and second lumens 131, 132 along with the first and second lumen openings 131A, 132A may be specifically configured for safe passage of blood therethrough. By way of example, a diameter of the first and second lumens 131, 132 may be sufficiently large to prevent damage (e.g., hemolysis) of blood passing therethrough at a flow rate of about 50, 100, 150 ml/min or more which hemolysis may be due to an elevated shearing rate and/or a substantial negative pressure, for example. Similarly, the first and second lumen openings 131A, 132A may include smooth corners and edges to prevent damage to the blood.

The frame 110 includes an anchoring mechanism 160 configured to secure the IOP 100 to the bone 50. In the illustrated embodiment, the anchoring mechanism 160 includes external threads 161 that enable to the IOP 100 to be screwed into the bone wall 51. Other anchoring mechanisms suitable for securing the IOP 100 to the bone 50, as may be contemplated by one of ordinary skill, such as barbs or an adhesive, for example, are also disclosed herein.

In some embodiments, during use, the IOP 100 may be oriented in relation to the bone 50. For example, the first and second cavities 121, 122 may be oriented longitudinally with respect to the bone 50. In such an orientation, IOP 100 may be oriented in relation to a flow direction of the bodily liquid 60 within the cavity 52 so that one of the first lumen opening 131A or the second lumen opening 132A are positioned upstream of the other one of the first lumen opening 131A or the second lumen opening 132A. By way of example, in the illustrated embodiment, the first lumen opening 131A is positioned upstream of the second lumen opening 132A. Such an orientation may be advantageous when the bodily liquid 60 extracted from the cavity 52 through the first lumen opening 131A and the bodily liquid 60 is returned to the cavity 52 via the second lumen opening 132A. Such an orientation may minimize recirculation of the bodily liquid 60 through the system 70. During use, the system 70 may extract the bodily liquid 60 (e.g., blood) at a rate of about 50 ml/min, 100 ml/min, 150 ml/min, or more and return the bodily liquid 60 at similar flow rates.

Some bones may be more suited for a given patient treatment using IOP 100 than other bones. For example, some bones may have a higher flow rate of blood through the cavity than other bones. Some embodiments of the IOP 100 may be specifically configured to couple with the humerus, such as the greater tubercle of the humerus, for example.

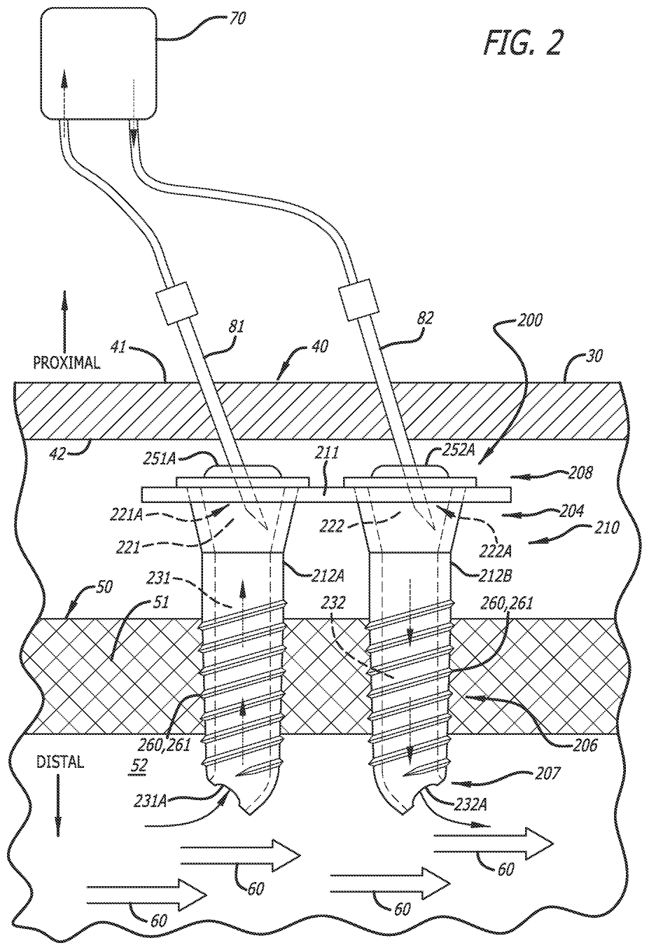

FIG. 2 illustrates a front elevation partial section view of a second embodiment of the IOP 200 that can, in certain respects, resemble components and functionality of the IOP 100 described in connection with FIG. 1. It will be appreciated that all the illustrated embodiments may have analogous features. Accordingly, like features are designated with like reference numerals, with the leading digits incremented to “2.” For instance, the frame is designated as “110” in FIG. 1, and an analogous frame is designated as “210” in FIG. 2. Relevant disclosure set forth above regarding similarly identified features thus may not be repeated hereafter. Moreover, specific features of the IOP and related components shown in FIG. 1 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the IOP of FIG. 2. Any suitable combination of the features, and variations of the same, described with respect to the IOP and components illustrated in FIG. 1 can be employed with the IOP and components of FIG. 2, and vice versa. This pattern of disclosure applies equally to further embodiments depicted in subsequent figures and described hereafter.

FIG. 2 shows the IOP 200 implanted within the patient 30 such that the IOP 200 is disposed entirely beneath the skin 40. The IOP 200 includes a proximal portion 204 disposed between the under surface 42 of the skin 40 and the bone 50. The IOP 200 is coupled with the bone 50 such that a distal portion 206 of the IOP 200 extends through the bone wall 51. A distal end 207 of the IOP 200 is disposed within the cavity 52. In some embodiments, the IOP 200 may configured such that a proximal end 208 of the IOP 200 is disposed adjacent the under surface 42 of the skin 40.

The IOP 200 includes a frame 210 that generally defines the structure of the IOP 200. The frame 210 may be formed of any rigid material suitable for implantation within the patient 30, such as stainless steel or plastic, for example. The frame 210 includes first and second tubular members 212A, 212B. The first tubular member 212A defines a first cavity 221 fluidly coupled with a first lumen 231 to define a first flow path extending between a first cavity opening 221A at the proximal end 208 and a first lumen opening 231A the distal end 207. Similarly, the second tubular member 212B defines a second cavity 222 fluidly coupled with a second lumen 232 to define a second flow path extending between a second cavity opening 222A at the proximal end 208 and a second lumen opening 232A at the distal end 207. The frame 210 further includes a connection member 211 configured to couple the first and second tubular members 212A, 212B together. Each of the first and second tubular members 212A, 212B are rotatably coupled with the connection member 211 so that the first and second tubular members 212A, 212B can rotate with respect to the connection member 211.

The IOP 200 includes a first septum 251A extending across the first cavity opening 221A and a second septum 252A extending across the second cavity opening 222A. The first and second septa 251A, 252A define a seal over the first and second cavity openings 221A, 222A, respectively. The first and second septa 251A, 252A are configured for piercing via a needles 81, 82 and resealing after the needles 81, 82 are removed. The first and second cavities 221, 222 are configured to receive tips of the needles 81, 82, respectively therein to provide access to the bodily liquid 60 therein. The first and second septa 251A, 252A may be formed of any suitable septum material configured for implantation within the body, such as EPDM, polytetrafluorethylene, Dacron, silicon, or polyurethane, for example. During use, the first and second septa 251A, 252A may be pierced several times (e.g., 5, 10, 20, 50 or more) over the life of the IOP 200.

Each tubular member 221A, 212B includes an anchoring mechanism 260 configured to secure the IOP 200 to the bone 50. In the illustrated embodiment, the anchoring mechanism 260 includes external threads 261 that enable to the tubular members 221A, 212B to be individually screwed into the bone wall 51. Other anchoring mechanisms suitable for securing the IOP 200 to the bone 50, as may be contemplated by one of ordinary skill, such as barbs or an adhesive, for example, are also disclosed herein.

In some instances of use, the IOP 200 may be oriented in relation to the bone 50. For example, the first and second cavities 221, 222 may be oriented longitudinally with respect to the bone 50. In such an orientation, IOP 200 may be oriented in relation to a flow direction of the bodily liquid 60 within the cavity 52 so that one of the first lumen opening 231A or the second lumen opening 232A are positioned upstream of the other one of the first lumen opening 231A or the second lumen opening 232A. By way of example, in the illustrated embodiment, the first lumen opening 231A is positioned upstream of the second lumen opening 232A. Such an orientation may be advantageous when the bodily liquid 60 extracted from the cavity 52 through the first lumen opening 231A and the bodily liquid 60 is returned to the patient via the second lumen opening 232A.

Furthermore, each tubular member 212A, 212B may be oriented in relation to the flow direction of a bodily liquid 60 within the cavity 52. As illustrated in FIG. 2, the first and second lumen openings 231A, 232A may be directed laterally with respect to its respective tubular member 212A, 212B. For example, in the illustrated embodiment, the first lumen opening 231A faces upstream with respect to the flow direction of the bodily liquid 60 and the second lumen opening 232A faces downstream with respect to the flow direction of the bodily liquid 60. Such an orientation of the first and second lumen openings 231A, 232A, may enhance the treatment process by minimizing recirculation of the bodily liquid 60 through the system 70.

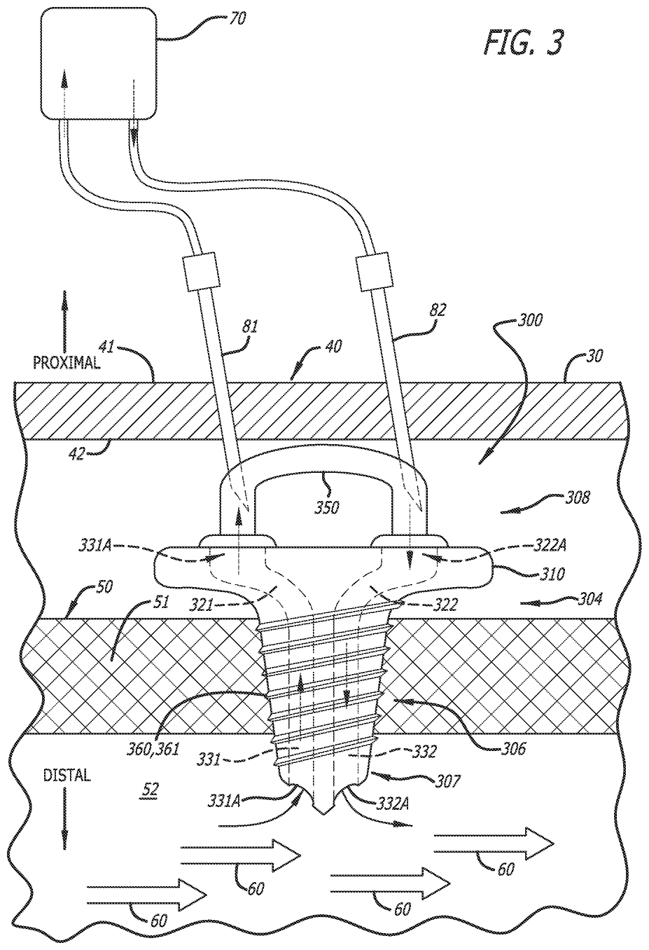

FIG. 3 illustrates a front elevation partial section view of a third embodiment of an intraosseous port (IOP) 300 in use with the patient 30 during a treatment defined by a medical system (system) 70. The IOP 300 is generally configured to be implanted within the patient, such as entirely within patient, for example. The IOP 300 is further generally configured to enable access to the bodily liquid 60. FIG. 3 shows the IOP 300 implanted within the patient 30 such that the IOP 300 is disposed entirely beneath the skin 40. The IOP 300 includes a proximal portion 304 disposed between the under surface 42 of the skin 40 and the bone 50. The IOP 300 is coupled with the bone 50 such that a distal portion 306 of the IOP 300 extends through the bone wall 51. A distal end 307 of the IOP 300 is disposed within the cavity 52.

The IOP 300 includes a frame 310 that generally defines the structure of the IOP 300. The frame 310 may be formed of any rigid material suitable for implantation within the patient 30, such as stainless steel or plastic, for example. The frame 310 defines a first cavity 321 fluidly coupled with a first lumen 331 to define a first flow path extending between a first cavity opening 321A at the proximal end 308 and a first lumen opening 331A at the distal end 307. Similarly, the frame 310 further defines a second cavity 322 fluidly coupled with a second lumen 332 to define a second flow path extending between a second cavity opening 322A at the proximal end 308 and a second lumen opening 332A at the distal end 307. In the illustrated embodiment, the first cavity 321 and first lumen 331 are positioned laterally with respect to the second cavity 322 and the second lumen 332 in a side-by-side relationship. In other embodiments, at least a portion of the first lumen 331 and the second lumen 332 may be positioned concentrically with respect to each other.

The IOP 300 includes a graft 350 extending between the first cavity opening 321A and the second cavity opening 322A, where the graft 350 defines a flow path between the first cavity 321 and the second cavity 322. The graft 350 is configured for piercing via the needles 81, 82 to provide access to the bodily liquid 60 therein. The graft 350 is further configured reseal after the needles 81, 82 are removed. In some embodiments, the first and second cavities 321, 322 are configured to receive the tips of the needles 81, 82, respectively therein. The graft 350 may be formed of any suitable graft material, such as polytetrafluorethylene, Dacron, silicon, or polyurethane, for example. During use, the graft 350 may be pierced several times (e.g., 5, 10, 20, 50 or more) over the life of the IOP 300.

The frame 310 includes an anchoring mechanism 360 configured to secure the IOP 300 to the bone 50. In the illustrated embodiment, the anchoring mechanism 360 includes external threads 361 that enable to the IOP 300 to be screwed into the bone wall 51.

A method 400 for treating a patient may include all or any subset of the following steps, actions, or processes. The method 400 may include coupling the intraosseous port to a bone of the patient (block 410). In some embodiments, coupling the intraosseous port to the bone includes implanting the intraosseous port subcutaneously within the patient. In some embodiments, coupling the intraosseous port to the bone includes threading the intraosseous port into the bone. In some embodiments, the bone may include the greater tubercle of a humerus.

The method 400 may include establishing a fluid connection between the medical system and the intraosseous port (block 420). In some embodiments, establishing the fluid connection may include (i) piercing the first septum of the intraosseous port with a first needle and (ii) piercing the second septum of the intraosseous port with a second needle. In some embodiments, the intraosseous port includes a graft coupled between the first lumen and the second lumen, and establishing a fluid connection includes piercing the graft with the first needle and piercing the graft with the second needle.

The method 400 may include extracting a first bodily liquid from the patient via the intraosseous port (block 430). In some embodiments, extracting a first bodily liquid includes drawing the first bodily liquid from a cavity (e.g., a medullary cavity) of the bone via the first lumen of the intraosseous port. In some embodiments of the method 400, extracting a first bodily liquid from the patient includes defining a flow rate of the first bodily liquid that exceeds about 50 ml/min, 100 ml/min, 150 ml/min, or more.

According to another embodiment of the method 400, extracting the first bodily liquid from the patient may include extracting the first bodily liquid from the patient via a route other than the intraosseous port, such as via a vascular catheter, for example.

The method 400 may include delivering a second bodily liquid to the patient via the intraosseous port (block 440). In some embodiments, delivering the second bodily liquid includes delivering the second bodily liquid to the cavity of the bone via the second lumen of the intraosseous port. In some embodiments, extracting the first bodily liquid and delivering the second bodily liquid occur simultaneously. In some embodiments, the second bodily liquid includes the first bodily liquid in a processed state in accordance with an operation of the medical system. According to another embodiment of the method 400, delivering a second bodily liquid to the patient may include delivering the second bodily liquid to the patient via a route other than the intraosseous port, such as via a vascular catheter, for example.

In some embodiments of the method 400, the medical system may be a hemodialysis system.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. An intraosseous port, comprising:
a frame configured for subcutaneous placement within a patient, the frame comprising:
a proximal portion having first cavity and a second cavity, the first and second cavities configured for fluidly coupling with a medical system and;
a distal portion configured for insertion through a bone wall, the distal portion including:
a first lumen extending along the distal portion between the first cavity and open distal end of the first lumen;
a second lumen extending along the distal portion between the second cavity and an open distal end of the second lumen, wherein the first and second open distal ends are configured for placement within a cavity of the bone; and
an anchoring mechanism configured for securing the frame to the bone.

2. The port according to claim 1, wherein the anchoring mechanism includes external threads.

3. The port according to claim 1, wherein the open distal end of the second lumen is spaced longitudinally away from the open distal end of the first lumen.

4. The port according to claim 1, wherein the second cavity is disposed laterally with respect to the first cavity.

5. The port according to claim 1, wherein the second lumen is disposed laterally with respect to the first lumen.

6. The port according to claim 2, wherein the anchoring mechanism includes a first threaded portion surrounding the first lumen and a second threaded portion surrounding the second lumen.

7. The port according to claim 1, further comprising:
a first septum extending across a proximal opening of the first cavity; and
a second septum extending across a proximal opening of the second cavity,
wherein the first and second septa are configured for passage of a needle therethrough.

8. The port according to claim 1, further comprising a graft extending between the first and second cavities.

9. The port according to claim 8, wherein the graft is configured to extend between the bone and an under surface of a skin of the patient.

10. The port according to claim 1, wherein the bone wall includes a greater tubercle of a humerus.

11. A method of treating a patient, comprising;
coupling an intraosseous port to a bone of the patient;
establishing a fluid connection between a medical system and the intraosseous port;
extracting a first bodily liquid from the patient via the intraosseous port; and
delivering a second bodily liquid to the patient via the intraosseous port.

12. The method according to claim 11, wherein coupling an intraosseous port to a bone of the patient includes implanting the intraosseous port subcutaneously within the patient.

13. The method according to claim 11, wherein:
extracting a first bodily liquid includes drawing the first bodily from a cavity of the bone via a first lumen of the intraosseous port; and
delivering a second bodily liquid includes delivering the second bodily liquid to the cavity of the bone via a second lumen of the intraosseous port.

14. The method according to claim 13, wherein extracting the first bodily liquid and delivering the second bodily liquid occur simultaneously.

15. The method according to claim 13, wherein the second bodily liquid includes the first bodily liquid in a processed state in accordance with an operation of the medical system.

16. The method according to claim 11, wherein coupling an intraosseous port to a bone of the patient includes threading the intraosseous port into the bone.

17. The method according to claim 13, wherein establishing a fluid connection between a medical system and the intraosseous port includes:
piercing a first septum of the intraosseous port with a first needle, and
piercing a second septum of the intraosseous port with a second needle.

18. The method according to claim 11, wherein:
the intraosseous port includes a graft extending between the first lumen and the second lumen, and
establishing a fluid connection between a medical system and the intraosseous port includes:
piercing the graft with a first needle, and
piercing the graft with a second needle.

19. The method according to claim 11, wherein:
the medical system is a hemodialysis system, and
the treatment includes hemodialysis.

20. The method according to claim 1, wherein extracting a first bodily liquid includes defining a flow rate of the first bodily liquid that exceeds about 50 ml/min.
